(19) United States
(12) Patent Application Publication
(10) Pub. No.: US 2013/0184816 A1
(43) Pub. Date: Jul. 18, 2013

(54) INTRAOCULAR LENS RINGS

(71) Applicant: Anew Optics, Inc., Bristol, TN (US)

(72) Inventor: Anna S. Hayes, Newton Centre, MA (US)

(73) Assignee: Anew Optics, Inc., Bristol, TN (US)

(21) Appl. No.: 13/729,767

(22) Filed: Dec. 28, 2012

Related U.S. Application Data
(60) Provisional application No. 61/581,855, filed on Dec. 30, 2011.

(51) Int. Cl. A61F 2/16

(52) U.S. Cl. CPC A61F 2/16
USPC 623/6.43

(57) ABSTRACT

The invention is directed to a lens that comprises an optic and at least two haptic rings, one positioned to rest against the posterior capsule distally outward from the optic zone, the other to rest on the anterior capsule some distance from the equator. The haptic rings of the lens are connected by segments of haptic material that may be arched or straight, and sections of open space to provide for ample circulation of the aqueous humor. The optic is suspended between the two haptic rings such that the distance between the optic and the anterior ring is constant while the distance between the optic and the posterior ring may vary according to the overall capsular dimensions of the eye of the patient.

### REFERENCE TO RELATED APPLICATIONS

This application claims priority to U.S. Provisional Application No. 61/581,855, filed Dec. 30, 2011, entitled “Intraocular Lens Rings,” which is specifically and entirely incorporated by reference.

### BACKGROUND

1. Field of the Invention

This invention is directed to intraocular lens and haptic devises that contain rings and, in particular rings that are designed to maintain a distance between the anterior and posterior capsule of the eye so as to allow circulation of the aqueous humor throughout the capsule.

2. Description of the Background

The natural lens of the human eye possesses certain features that are critical to the importance of the inventive lens. The natural eye capsule is generally ovate in shape when seen from the side, and essentially circular in shape when viewed through the cornea. The imputed radius of curvature of the anterior capsule of the lens is greater than the imputed radius of curvature of the posterior capsule, and though the ratio of the radii of curvature changes if the eye is measured in a distance vision state or in a near vision (or accommodative) state, the absolute arc length of the anterior and posterior capsule segments do not change.

The capsule is retained in the eye by means of a network of zonules, which are fixed length fibers that do not demonstrate elasticity, and are attached to the capsule at various points anterior to, posterior to, and within the fornix (or equatorial zone) of the eye, and at the other end to the ciliary body at certain point(s) posterior to the ciliary point. When the ciliary body expands anteriorly and toward the pupil, the zonules relax tension on the capsule and the capsule becomes more circular in shape, thus providing a greater diopter power for near vision accommodation. When the ciliary body retracts posteriorly and away from the pupil, the zonules exert force on the capsule, stretching the capsule outward and making a flatter ovate shape, thus providing for distance vision. It is important within the context of the inventive haptic design to note that the forces of the ciliary body and the changes in pressure of the zonules resulting in the changes in curvature of the lens capsule are primarily such that the anterior capsul4 moves toward the iris. The nodal point of the posterior capsule remains in place, and the central area of the posterior capsule is not believed to change significantly. This is a component of the natural physics of the eye, that seeks to maintain suitable positive pressure of the vitreous on the retina to avoid tendency toward retinal detachment. The natural lens capsule at the equator varies in diameter between approximately 8.9 millimeters in the accommodative state to approximately 9.5 millimeters in the distance state. All measurements are approximate as each eye varies in size and capsular dimensions can change over time, particularly when considering the juvenile eye.

While the angles of the zonules to the ciliary point are most variable in accommodation in the most anterior and posterior sets of zonules, the zonules closest to the fornix are likely to demonstrate least change in accommodation thus greatest stability in maintaining the correct position of the capsule in the eye.

A third set of zonules attaches the ciliary body to a series of points through the vitreous. It is believed that the third set of zonules is fundamental in maintaining correct positioning of the capsule in the eye and preventing excess anterior dislocation of the capsule in accommodation. This also provides for constant position of the central portion of the posterior capsule, as indicated above.

Most intraocular lenses currently available are essentially two-dimensional, and consist of an optic centrally located between plate, c-loop, or other haptics and with overall lens diameter of approximately 11 millimeters. The lens diameter is, notably, longer than the natural diameter of the lens capsule, as the purpose of these conventional lens designs is to stretch the capsule to an essentially flattened shape. Flattening the capsule can have significant negative consequences. Firstly, flattening pulls the posterior capsule forward in the eye which may create negative pressure on the vitreous and increase the risk of detachment of the retina. Secondly, these lenses generally do not have haptics that preserve the circular configuration of the capsule at the fornix, which means that the relationship between the capsule and the zonules is disrupted which may cause undue stress on the zonules and on the ciliary body. Thirdly, these lenses generally cause the capsule to fibrose over time, and those sections of the capsule that are not separated by the lens tend to adhere to each other. Fourthly, contact between the capsule and the lens, both anterior and posterior, predisposes the eye to develop anterior and posterior capsular opacification which can ultimately cloud the optical area of the lens and, without additional surgery, diminish the eyesight of the patient. After cataract surgery within the capsule that contained the natural lens are cells similar to the cells that initially grew the cataractous condition. The cells migrate from along the anterior surface of the lens to the equator where they die, releasing a protein blast of lens cortical material, that accumulates with other cortical material to form a blanket, a pattern of fibrous strands, or other formations, that then move posteriorly along the natural lens capsule until the density is such that light traveling through the lens is impeded, impairing the patient's vision. Laser light is used to split the posterior portion of the capsule in a procedure labeled an Nd_YAG laser posterior capsulotomy to open up the optical zone of the posterior capsule so as to allow the patient's vision to be restored. Rupture of the posterior capsule has significant attendant risks, including possible prolapse of the vitreous into the anterior segment of the eye, which could require additional surgical procedures.

Most intraocular lenses have optics that range in diameter from 5 millimeters to 6 millimeters; in rare occasions are lens optics greater than 6 millimeters, as the greater diameter tends to cause too much bulk to allow insertion of the lens into the eye through an incision of less than 3 mm. Because the pupil of the eye is not centered in the eye but located approximately 5° toward the nose, some intraocular lens recipients complain that they have a “blind spot” which is generally thought to result from the patient perceiving the edge of the optic, particularly with lenses with optics of 5 mm or less. This condition can become exacerbated by the position of the lens in the eye; the anterior the lens is positioned, the greater the distance from the lens to the retina, therefore the higher risk that the edge of the lens could be visible, especially when the pupil is dilated (such as when driving at night).

Thus, a need exists for an intraocular lens that is less bulky and provides for accommodative correction, yet preserves as much as possible the natural configuration of the eye capsule.

### SUMMARY

The present invention overcomes the problems and disadvantages associated with current strategies and designs, and provides new tools and methods for lens replacement therapies.

One embodiment of the invention is directed to an optic, preferably at least 6.5 mm in size, positioned between anterior and posterior rings such that the optic rests at a point that is close to the nucleus of the natural lens for depth of field and suitable focal power.

Another embodiment of the invention is directed to one or more haptic pillars positioned between the optic ring and the anterior ring that are essentially perpendicular to the anterior and optic rings so as to preserve the distance between the optic and the anterior capsule. The distance maintains the A-Constant measurement of the lens optic, thereby assuring suitable and consistent visual acuity for the patient.

Another embodiment of the invention is directed to one or more haptic supports positioned between the posterior ring and the optic ring that are constructed at an angle such that the distance between the posterior ring and the optic ring may vary according to the size of the capsule. The compressibility of the haptic supports are particularly important in the case of juvenile eyes, whose capsular circumference may be smaller at the time of cataract surgery but may be expected to increase in circumference with aging. The adjustable distance between the optic ring and the posterior ring can change with changes in the size of the capsule, thus providing for long lasting fit and functionality in the eye. The configuration of the posterior haptic supports also provides for the inventive lens to respond to prompts of the ciliary body such that, when the ciliary body moves toward the iris so as to provide accommodation, the posterior haptic pillars will decompress, thus allowing the distance between anterior and posterior rings to increase, moving the optic forward in the eye for the focal accommodative effect.

Another embodiment of the inventive lens is in the full circular nature of the haptic rings, such that the anterior ring can arrest lens epithelial cells that are migrating toward the fornix along the anterior capsule, and these cells, when they detach, will not reattach, but will be washed away by the aqueous humor to the trabecular meshwork and out of the eye. This minimizes, over time, the number of lens epithelial cells that can reach the fornix, thereby controlling the extent to which lens corticular material can be formed. The posterior ring serves to form a barrier around the perimeter of the optical zone of the posterior capsule, such that any lens corticular material may not encroach into the optical zone, thus preserving the patient's quality of vision and avoiding secondary clouding of the capsule. The posterior ring also provides a mechanism whereby the lens corticular proteins may be dislodged from the capsular surface and washed away by the aqueous humor.

Another embodiment of the inventive lens is in the ample fenestration of the haptic columns such that the aqueous may circulate throughout the entire capsule. The fenstration may be instrumental in preventing fibrosis of the capsule.

One embodiment of the invention is directed to a compressible haptic device for an intraocular lens. The haptic device comprises a haptic body, an anterior ring positioned anteriorly to the haptic body and at a first distance from the haptic body. at least one ridged haptic pillar coupling the haptic body to the anterior ring, a posterior ring positioned posteriorly to the haptic body and at a second distance from the haptic body, and at least one compressible haptic support coupling the haptic body to the posterior ring.

Preferably the haptic body, the anterior ring, the at least one haptic pillar, the posterior ring, and the at least one haptic support are composed of the same material. In a preferred embodiment, the at least one haptic pillar or the at least one haptic support is solid or perforated. The haptic device preferably further comprises an optic that is suspended from the haptic body. Preferably, the optic is plano to the haptic body.

Preferably, the optic is vaulted anterior or posterior to the optic center of the eye and remains fully contained within the anterior and posterior rings. Preferably, the haptic device further comprises perforations between the outer diameter of the optic and the haptic body. In a preferred embodiment, the diameter of the optic is at least 6.5 mm. Preferably, the haptic body is shaped as a ring.

In a preferred embodiment, the anterior ring has a diameter that is larger or smaller than the posterior ring. Preferably, the distance between anterior ring and the posterior ring is at least 4 mm. Preferably, the anterior ring and the posterior ring each possesses interior grooves and said interior grooves face each other. In a preferred embodiment, one or more additional optics are affixed to the interior grooves.

Another embodiment of the invention is directed to a single-piece intraocular lens. The lens comprises an anterior ring that contacts a lens capsule at a distance anterior to the fornix of the lens capsule, a posterior ring that rests against the lens capsule at a distance posterior to the fornix of the lens capsule, a haptic segment coupling the anterior ring to the posterior ring, and an optic centered posterior to the posterior ring so as to be positioned at the deepest natural point of the lens capsule.

Preferably, the lens is comprised of a hydrophilic acrylic, a hydrophobic acrylic, a silicone, PMMA, a polymer, or a combination thereof. In a preferred embodiment, the lens is composed of more than one type of material. Preferably, the optic is at least 6.5 mm in diameter. Preferably, the haptic segment is at least partially compressible. In a preferred embodiment, the haptic segment contains at least one perforation. Preferably the anterior ring has a greater or a lesser diameter than the posterior ring. Other embodiments and advantages of the invention are set forth in part in the description which follows, and in part, may be obvious from this description, or may be learned from the practice of the invention.

### DESCRIPTION OF THE FIGURE

FIG. 1 depicts one embodiment of the lens of the invention from the side view.

### DESCRIPTION OF THE INVENTION

The lens of the invention addresses key considerations within the field of ophthalmology and specifically within the field of lens replacement surgery, whether for cataracts, presbyopia correction, or other medical needs. The invention addresses the importance of maintaining the capsule in an open configuration while providing for differences in capsular circumference.

This invention is generally directed to intraocular lens and haptic devises that contain rings and, in particular rings that are designed to maintain a distance between the anterior and posterior capsule of the eye so as to allow circulation of the aqueous humor throughout the capsule. The anterior ring is designed to maintain a constant distance between the optic and the cornea of the eye, which provides consistent refractive power in an eye regardless of the size of the capsule. The posterior ring is designed to rest against the posterior capsule at a distance from the equator of the capsule and is structured so as to be able to move anteriorly toward the optic and anterior ring in the event that the capsule is smaller than average, or when the capsule is in a distance vision state. This ensures that the lens can adapt to various dimensions of eye capsules without requiring custom sizing.

One embodiment of the invention is directed to a lens that comprises an optic and two haptic rings, one positioned to rest against the posterior capsule distally outward from the optic zone, the other to rest on the anterior capsule some distance from the equator. The haptic rings of the lens are connected by segments of haptic material that are preferably straight between the a haptic body coupled to the optic and the anterior ring, so as to provide for structural strength and stiffness in that segment of the inventive lens, and segments of haptic material between the haptic body and the posterior ring that may be arched or elbowed so as to provide for structural responsiveness to differences in capsular dimensions, and sections of open space to provide for ample circulation of the aqueous humor.

In the first instance, the anterior ring is located anterior of the fornix and the fornix does not contact the haptic ring but is held in place by the relationship between the anterior and posterior haptic rings. A function of the haptic rings is to keep the capsule configured as naturally as possible, while the anterior ring is designed to arrest the migration of lens epithelial cells along the anterior capsule to the fornix, thereby mitigating the onset or occurrence of Anterior Capsule Opacification (ACO), and the posterior ring is positioned on the posterior capsule to prevent incursion of lens cortical material into the optical zone in the form of PCO (Posterior Capsular Opacification). The haptic perforations are designed to enhance circulation of the aqueous humor throughout targeted areas of the capsule so as to preserve overall capsular hydration and health.

The lens of the invention preferably blocks cell migration and prevents posterior capsular opacification. The lens preferably has an anterior surface with rings and a posterior surface. The posterior surface preferably has an anterior ring that prevents or blocks cells from migrating from along the anterior portion of the natural lens toward the capsular equator. The posterior ring blocks cells from the equatorial zone from passing toward the optical portion of the lens. Between the anterior and posterior ring preferably there are slots cut into the haptic portion of the lens to allow aqueous flow throughout the capsule. The aqueous flow will allow cells in the equatorial zone to be flushed from the cavity and carried out of the eye by the natural fluid flow system of the eye.

The optic is preferably designed to be positioned anterior of the posterior capsule but posterior of the fornix of the capsule. The functionality of the location of the optic is important in at least three respects: first, the position between the optic and the anterior ring is maintained so as to assure long term refractive consistency; second, the optic is positioned as close as possible to the center of the natural lens, which provides for optical fidelity, thus enhancing the patient's quality of vision; third, the size of the optic diameter at about 6.5 mm mitigates significantly any potential risk that the patient's vision will be disrupted by being able to discern the edge of the optic.

The entire lens, haptic and optic assembly, is preferably configured and designed so as to be able to be inserted in the human eye through an incision of preferably less than about 3 mm. Lens replacement surgery is preferably performed using very small incisions, reducing thereby the trauma to the patient and mitigating the need for sutures.

The following examples illustrate embodiments of the invention, but should not be viewed as limiting the scope of the invention.

### EXAMPLES

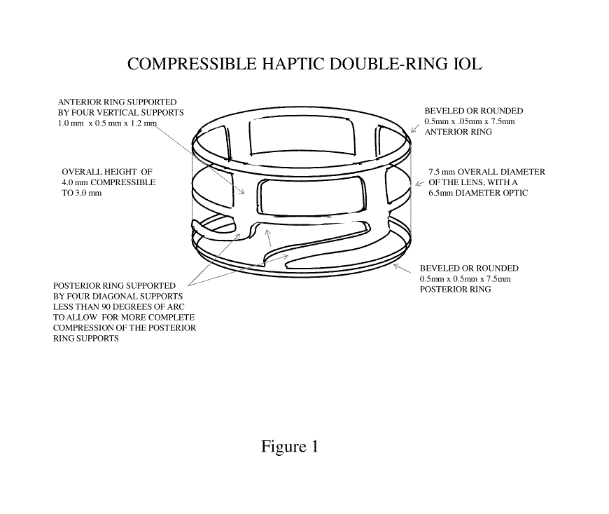

FIG. 1 depicts one embodiment of the lens of the invention from the side view, demonstrating the anterior and posterior rings and the location of the inventive optic at a fixed distance from the anterior haptic ring. In this figure the relative dimensions of the anterior and posterior rings are designed such that the rings are essentially equal in radius, though the posterior ring may be configured so as to be smaller than, equal to, or larger in radius than the anterior ring. The haptic pillars that connect the anterior and posterior rings to the ring containing the optic are designed such that the distance between the optic and the anterior ring remains constant while the distance between the optic and the posterior ring can decrease by as much as about 1 mm so as to allow the lens to adapt to different capsular circumferences.

Another embodiment of the invention is directed to a lens comprising a fully circular, three-dimensional design comprised of four key components: an anterior haptic ring, a center haptic ring (or haptic body) to which the optic is connected, a posterior haptic ring, a series of pillars connecting the anterior ring to the haptic body, and a series of haptic structures connecting the posterior ring to the haptic body. Preferably the lens is composed of hydrophilic or hydrophobic acrylic, though in certain embodiments the lens and rings may also be made of silicone or such other material as is suitable for insertion into the eye. Preferably each of the haptic rings has an overall diameter of about 7.5 mm, and the lens has an optic diameter of about 6.5 mm. The distance between the anterior ring and the posterior ring is preferably about 4 mm with the haptic body equidistant from both outer rings. Preferably the haptic pillars between the anterior ring and the haptic body are configured so that the distance between the haptic body and the anterior ring remains constant. Preferably the anterior and posterior rings are beveled or rounded with a ring diameter of about 500 microns or less. The anterior to haptic body pillars preferably are about 1 mm wide and about 500 microns thick, and approximately 1.2 mm in height, and there are preferably three, four, five, six or more haptic pillars. The optic to posterior haptic structures preferably are configured as elbows that may compress up to about 1 mm such that the overall height of the lens, preferably about 4.0 mm may be compressed to about 3.0 mm, thus accommodating to fit lens capsules with overall circumference of about 21 mm to 23 mm.

This lens design addresses concerns expressed by ophthalmologists with respect to the successful implantation of an aphakic lens following cataract or refractive lens replacement surgery, specifically those concerns related to the size of the capsule both at and subsequent to surgery, and the ability to maintain capsular configuration as much as possible without placing any undue stress on the zonules.

Most aphakic lenses currently approved for cataract or other lens replacement surgical procedures are essentially two-dimensional and stretch the capsule toward the fornix (equator). This has the effect of flattening the lens capsule out, specifically of stretching the anterior capsule flat and of stretching the posterior capsule flat at a location in the lens proximate to the fornix. Over time the lens capsule can fibrose, becoming leathery, and shrink in overall size. The effect of this shrinkage is to pull the capsule away from the ciliary body. The entire reconfiguration of the lens capsule can also place negative pressure on the retina, possibly increasing the risk of retinal detachment. The lens of the invention is preferably a three-dimensional, fully circular design that is intended to be centered in the capsule at some distance from the fornix, such that the anterior ring haptic rests on the inner surface of the anterior capsule, and the posterior ring haptic rests against the inner surface of the posterior capsule, keeping the capsule fully three-dimensional, thus more in its natural configuration. By maintaining an open capsule the inventive design allows circulation of the aqueous humor around the lens and throughout the capsule. A significant benefit of capsular hydration and aqueous circulation is in the maintenance of capsular health and the prevention of fibrosis and consequent capsular atrophy or shrinkage. Thus, the lens design of the invention preserves the overall size of the capsule post surgery, which has lasting benefits to the patient in maintaining focal flexibility and overall quality of sight. In addition, the posterior ring of the inventive lens keeps the posterior capsule as close as possible to the retina, thus avoiding risk of retinal detachment due to the surgical procedure or lens design.

Different patients have significant variation in capsular size, the specific actual dimensions being difficult to measure accurately prior to surgery, thus there is a need for aphakic lenses that can adapt to different capsule sizes. This is particularly remarkable in any of the lenses that purport to use some of the natural physical dynamics of the eye to provide focal accommodation. While many conventional lenses are positioned in the capsule by means of flexible, c-loop haptics, thus able to fit in various size eye capsules, lenses that are three-dimensional in design with full anterior and posterior rings address capsular dimensions either through providing different sized lenses to the ophthalmologist, which may or may not be successful depending upon the accuracy with which the ophthalmologist can measure the patient's capsule prior to surgery, or through built-in elasticity. The inventive lens addresses different capsular sizes by creating diagonal apertures in the haptic by designing haptic pillars shaped like elbows and wrists, or knees and ankles posterior to the circular plane supporting the optic. In this fashion, the posterior haptic adjusts in overall height to accommodate smaller eye capsules. Furthermore, the haptic pillars of the invention are anterior to the optic plane have rectangular apertures, which ensure that the distance from the anterior capsule to the optic remains the same in all size eyes, thus providing an enhancement in A-constant measurement on an intracapsular basis. This helps to ensure that the patient receives the proper optical power in the lens. Moreover, preferably in larger eyes, the optic is positioned in close proximity to the nucleus of the natural lens, which provides both suitable depth of field and good visual clarity. The optic is preferably positioned so as to be plano, convex, aspheric, Fresnel, concave, or any combination of these configurations, so as to provide suitable optical power and clarity for the intended application.

FIG. 1 sets forth the preferable dimensions of the Compressible Haptic Double-Ring IOL, with an illustration of the compressible rings and the haptic pillars connecting the rings. Preferably the interior of the anterior ring contains an angular inner edge and a beveled or rounded outer edge. One function of the angled inner edge is to arrest lens epithelial cells as they migrate along the anterior capsule. The function of the beveled or rounded outer edge is to conform the ring to the anterior capsule so as to provide a suitable surface of contact with the anterior capsule yet mitigate the risk of perforating or tearing the capsule in any way. Preferably also the outer contour of the posterior haptic ring is beveled or rounded, so as to conform to the contour of the lens capsule. The junction of the vertical portion of the posterior ring and the bevel may preferably have an angle so as to mitigate any migration of Soemmering's Rings into the posterior capsule optical zone.

Note that once the implantation is complete, the anterior capsule will flatten somewhat as will the posterior capsule, but the intention of the inventive design is to preserve the natural contour of the fornix so as to maintain equilibrium in the natural relationship of the lens capsule to the zonules and the ciliary body. Thus, with the device of the inventive lens provides sufficient support to maintain an open capsule but at the same time provides for sufficient elastic response to allow the fornix to control the anterior/posterior dimension of the lens so as to maintain a space between the lens and the fornix and thereby preserve the natural curvature of the fornix of the natural lens.

Other embodiments and uses of the invention will be apparent to those skilled in the art from consideration of the specification and practice of the invention disclosed herein. All references cited herein, including all publications, U.S. and foreign patents and patent applications, are specifically and entirely incorporated by reference. The term comprising, where ever used, is intended to include the terms consisting and consisting essentially of. Furthermore, the terms comprising, including, and containing are not intended to be limiting. It is intended that the specification and examples be considered exemplary only with the true scope and spirit of the invention indicated by the following claims.

1. A compressible haptic device for an intraocular lens comprising:
a haptic body;
an anterior ring positioned anteriorly to the haptic body and at a first distance from the haptic body;
at least one ridged haptic pillar coupling the haptic body to the anterior ring;
a posterior ring positioned posteriorly to the haptic body and at a second distance from the haptic body; and
at least one compressible haptic support coupling the haptic body to the posterior ring.

2. The haptic device of claim 1, wherein the haptic body, the anterior ring, the at least one haptic pillar, the posterior ring, and the at least one haptic support are composed of the same material.

3. The haptic device of claim 1, wherein the at least one haptic pillar or the at least one haptic support is solid or perforated.

4. The haptic device of claim 1, further comprising an optic that is suspended from the haptic body.

5. The haptic device of claims 4, wherein the optic is plano to the haptic body.

6. The haptic device of claim 4, wherein the optic is vaulted anterior or posterior to the optic center of the eye and remains fully contained within the anterior and posterior rings.

7. The haptic device of claim 4, further comprising perforations between the outer diameter of the optic and the haptic body.

8. The haptic device of claim 4, wherein the diameter of the optic is at least 6.5 mm.

9. The haptic device of claim 1, wherein the haptic body is shaped as a ring.

10. The haptic device of claim 1, wherein the anterior ring has a diameter that is larger or smaller than the posterior ring.

11. The haptic device of claim 1, wherein the distance between anterior ring and the posterior ring is at least 4 mm.

12. The haptic device of claim 1, wherein the anterior ring and the posterior ring each possesses interior grooves and said interior grooves face each other.

13. The haptic device of claim 12, wherein one or more additional optics are affixed to the interior grooves.

14. A single-piece intraocular lens comprising:
an anterior ring that contacts a lens capsule at a distance anterior to the fornix of the lens capsule;
a posterior ring that rests against the lens capsule at a distance posterior to the fornix of the lens capsule;
a haptic segment coupling the anterior ring to the posterior ring; and
an optic centered posterior to the posterior ring so as to be positioned at the deepest natural point of the lens capsule.

15. The lens of claim 14, which is comprised of a hydrophilic acrylic, a hydrophobic acrylic, a silicone, PMMA, a polymer, or a combination thereof.

16. The lens of claim 14, which is composed of more than one type of material.

17. The lens of claim 14, wherein the optic is at least 6.5 mm in diameter.

18. The lens of claim 14, wherein the haptic segment is at least partially compressible.

19. The lens of claim 14, wherein the haptic segment contains at least one perforation.

20. The lens of claim 14, wherein the anterior ring has a greater or a lesser diameter than the posterior ring.
